(19) United States
(12) Patent Application Publication
(10) Pub. No.: US 2020/0275915 A1
(43) Pub. Date: Sep. 3, 2020

(54) ULTRASOUND PROBE LOCALIZATION WITH DRIFT CORRECTION

(71) Applicant: KONINKLIJKE PHILIPS N.V., EINDHOVEN (NL)

(72) Inventors: Jochen Kruecker, Andover, MA (US); Faik Can Meral, Mansfield, MA (US)

(21) Appl. No.: 16/645,684

(22) PCT Filed: Aug. 28, 2018
(86) PCT No.: PCT/EP2018/073062

§ 371 Date: Mar. 9, 2020

Related U.S. Application Data
(60) Provisional application No. 62/555,743, filed on Sep. 8, 2017.

(51) Int. Cl. A61B 8/08; A61B 34/20; A61B 8/00; G06T 7/33

(52) U.S. Cl. CPC A61B 8/5276; A61B 34/20; A61B 8/483; A61B 8/4254; G06T 2207/30004; A61B 8/5246; G06T 7/337; A61B 2034/2048; G06T 2207/10136; A61B 8/467

(57) ABSTRACT

An ultrasound (US) device (10) includes a US scanner (14) and a US probe (12) operatively connected to the US scanner. The US scanner and US probe are configured to acquire a succession of two-dimensional (2D) image frames of a portion of a patient. The device also includes a tracking sensor (28); and at least one electronic processor (20) programmed to: acquire a reference three-dimensional (3D) image using the US scanner and US probe; track the pose of each 2D image frame relative to the pose of the last 2D image frame in the succession of 2D image frames based on tracking data acquired by the tracking sensor; and correct for drift of the trucked poses of the succession of 2D image frames by drift correction operations including: aligning a current 2D image frame with the reference 3D image to optimize a similarity metric measuring similarity of the current 2D image frame to an intersected slice of the reference 3D image; and updating the pose of the current 2D image frame to a corrected pose determined from the alignment of the current 2D image frame with the reference 3D image.

### FIELD

The following relates generally to ultrasound imaging arts, ultrasound probe tracking arts, ultrasound probe drift correction arts, and related arts.

### BACKGROUND

In medical procedures, real-time information about the spatial position and orientation (i.e. the “pose”) of a medical device is often required. Typically, such information is obtained using optical, electro-magnetic or ultrasound tracking systems. Such systems are expensive and sometimes require significant setup time and effort. For some procedures, the device to be tracked is an imaging device (e.g. ultrasound probe), and it is desirable to provide the tracking information at the lowest possible cost. One example for such a procedure is ultrasound-guided prostate biopsy, in particular ultrasound-MRI fusion biopsy.

Lower-cost solutions for ultrasound tracking commonly involve image-based tracking and inertial sensors such as gyroscopes and accelerometers attached to the ultrasound probe.

Low-cost-sensors combined with image-based position estimates can achieve tracking accuracies of less than 3 mm for brief scans and simple scan geometries such as a uni-directional “sweep” across an organ that is required to reconstruct a three-dimensional (3D) view of that organ.

However, for longer scans and complex scan geometries, including arbitrary and multiple probe rotations as commonly encountered in free-hand ultrasound scanning, accuracy of such low-cost tracking solutions will deteriorate and are not clinically acceptable. Small errors or bias in the frame-to-frame pose measurements and calculations accumulate, leading to deteriorating pose estimates over time. Therefore, low-cost tracking is currently only suitable to perform brief scans with simple scan geometries, but is less well-suited for more complex clinical scan geometries.

The following discloses new and improved systems and methods.

### SUMMARY

In one disclosed aspect, an ultrasound (“US”) device includes a US scanner and a US probe operatively connected to the US scanner. The US scanner and US probe are configured to acquire a succession of two-dimensional (2D) image frames of a portion of a patient. The device also includes a tracking sensor, and at least one electronic processor programmed to: acquire a reference three-dimensional (3D) image using the US scanner and US probe; track the pose of each 2D image frame relative to the pose of the last 2D image frame in the succession of 2D image frames based on tracking data acquired by the tracking sensor; and correct for drift of the tracked poses of the succession of 2D image frames by drift correction operations including: aligning a current 2D image frame with the reference 3D image to optimize a similarity metric measuring similarity of the current 2D image frame to an intersected slice of the reference 3D image; and updating the pose of the current 2D image frame to a corrected pose determined from the alignment of the current 2D image frame with the reference 3D image.

In another disclosed aspect, a non-transitory computer readable medium stores instructions executable by at least one electronic processor to perform a drift correction method. The method includes: acquire two-dimensional (2D) images using a US scanner and a US probe; acquire a reference three-dimensional (3D) image using the US scanner and the US probe; track a pose of each 2D image frame relative to the pose of the last 2D image frame in the succession of 2D image frames based on tracking data acquired by a tracking sensor; and correct for drift of the tracked poses of the succession of 2D image frames by drift correction operations including: aligning a current 2D image frame with the reference 3D image to optimize a similarity metric measuring similarity of the current 2D image frame to an intersected slice of the reference 3D image; and updating the pose of the current 2D image frame to a corrected pose determined from the alignment of the current 2D image frame with the reference 3D image.

In another disclosed aspect, an ultrasound (US) device includes a US scanner and a US probe operatively connected to the US scanner. The US scanner and US probe are configured to acquire a succession of two-dimensional (2D) image frames of a portion of a patient. A tracking sensor includes an inertial sensor tracking relative changes in the position of US probe in which the inertial sensor including one of a gyroscope or an accelerometer. At least one electronic processor is programmed to: acquire a reference three-dimensional (3D) image using the US scanner and US probe; track the pose of each 2D image frame relative to the pose of the last 2D image frame in the succession of 2D image frames based at least in part on the relative changes in the position of the US probe tracked by the inertial sensor; and correct for drift of the tracked poses of the succession of 2D image frames by drift correction operations including: aligning a current 2D image frame with the reference 3D image to optimize a similarity metric measuring similarity of the current 2D image frame to an intersected slice of the reference 3D image; and updating the pose of the current 2D image frame to a corrected pose determined from the alignment of the current 2D image frame with the reference 3D image. The pose of the 2D image frame following the current 2D image frame in the succession is tracked relative to the updated pose of the current 2D image frame.

One advantage resides in providing a low cost US probe tracking device.

Another advantage resides in correcting for errors in frame to frame pose measurements of a US probe relative to a patient.

Another advantage resides in providing for use of image-based and/or inertial sensor-based US probe tracking with improved performance for longer scans and/or complex free-hand probe manipulations.

Another advantage resides in performing intermittent or continuous image-based registrations to correct for accumulating tracking errors of the pose of a US probe live ultrasound (US) imaging in the context of a baseline 3D-US image and/or an earlier-acquired 3D-MRI or other planning image, with improved correction of the baseline 3D-US or 3D-MRI image for tissue motion that may have occurred before or during the image-guided surgical procedure.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically shows an illustrative ultrasound (US) device in accordance with one aspect.

FIG. 2 shows an exemplary flow chart operation of the device of FIG. 1;

FIG. 3 shows another exemplary flow chart operation of the device of FIG. 1; and

FIG. 4 shows images with a corrected pose generated by the device of FIG. 1.

### DETAILED DESCRIPTION

The following relates to ultrasound tracking of interventional procedures. More costly ultrasound based tracking employs electromagnetic (“EM”) tracking of an ultrasound (US) probe to provide an absolute spatial reference. To the contrary, in lower-cost ultrasound tracking approaches disclosed herein it is desired to eliminate the relatively costly EM tracker and either employ only US tracking or US in combination with lower-cost relative tracking devices such as gyroscopes and/or accelerometers. Such tracking provides relative position, i.e. change of position, but not absolute positioning.

A problem arises in that since the tracking only determines positional change between successive US frames, cumulative error can build up leading to increasingly inaccurate positioning relative to the first frame of the series.

In embodiments disclosed herein, a pre-operative 3D ultrasound reference image is acquired. When drift correction is desired, the current 2D ultrasound image frame is initially registered to the previous 2D ultrasound image frame to provide an approximate initial alignment, and then the frame is aligned to the 3D reference image so as to optimize a similarity metric measuring similarity of the frame to the intersected slice of the 3D reference image. The pose of the current frame is then adjusted to line up with this fitted pose, and becomes the new “initial” frame for subsequent relative US tracking.

The drift correction may be variously triggered, e.g. manually by the surgeon when he or she suspects the tracking has large error, or based on detection of an operation such as probe rotation that is likely to introduce substantial error, or by performing a fast computation of the similarity metric and triggering a drift correction upon the similarity metric value degrading past some correction trigger threshold.

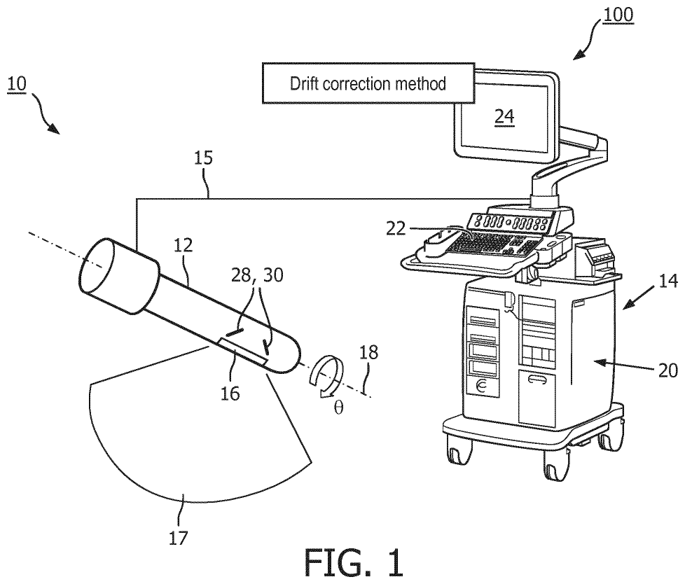

With reference to FIG. 1, an illustrative interventional imaging device suitable for implementing the foregoing is shown. An ultrasound (US) imaging device 10 may, for example, be an EPIQ™ ultrasound imaging system available from Koninklijke Philips N.V., Amsterdam, the Netherlands, a UroNav® system for US/MRI-fusion-guided prostate biopsy available from Koninklijke Philips N.V., Amsterdam, the Netherlands, the PercuNav® system (available from Koninklijke Philips N.V., Amsterdam) for general fusion of US with prior 3D imaging (CT, MR, cone-beam CT, etc.), or may be another commercial or custom-built ultrasound imaging system. The ultrasound imaging device 10 includes an US probe 12 operatively connected to an US scanner 14 to perform ultrasound imaging. The illustrative ultrasound probe 12 is connected with the ultrasound imaging system 10 via cabling 15, though a wireless connection is contemplated. The US probe 12 includes a sensor array 16 that acquires a two-dimensional (2D) image frame in a sonicated plane 17. The surgeon or other operator can adjust the location and orientation (i.e. “pose”) of the image frame by free-hand movement of the ultrasound probe 12. Such free-hand motion may entail a translational sweep of the US probe 12 (and hence of the sonicated plane 17) and/or may include rotating the US probe 12 about an axis 18, e.g. through an angle .theta (θ). The US scanner 14 and the US probe 12 are configured to acquire a succession of two-dimensional (2D) image frames of a portion of a patient, where each 2D image frame corresponds to the current pose of the sonicated plane 17.

The US scanner 14 also includes at least one electronic processor 20 (e.g., a microprocessor, a microcontroller, and the like), at least one user input device (e.g., a mouse, a keyboard, a trackball, and/or the like) 22, and a display 24 for displaying ultrasound images and/or US scanner settings, image parameters, and/or so forth.

The at least one electronic processor 20 is operatively connected with a non-transitory storage medium (not shown) that stores instructions which are readable and executable by the at least one electronic processor 20 to perform disclosed operations including, e.g. operating the US scanner 14 to perform live US imaging and performing a drift correction method or process 100 to correct a position of the US probe 12 relative to the portion of the patient being scanned. The non-transitory storage medium may, for example, comprise a hard disk drive or other magnetic storage medium; a solid state drive (SSD), flash drive, electronically erasable read-only memory (EEROM) or other electronic memory; an optical disk or other optical storage; various combinations thereof; or so forth. In some embodiments the non-transitory storage medium storing the instructions is disposed in the US scanner 14, e.g. in the form of an internal hard drive, SSD, flash memory, and/or the like.

In some embodiments, the US imaging device 10 includes at least one tracking sensor 28 disposed on a portion of the US probe 12. The tracking sensor 28 is configured to track orientation of the US probe 12. In some examples, the tracking sensor 28 comprises the US scanner 14 and the US probe 12. In other examples, the tracking sensor 28 comprises an inertial sensor 30 configured to track relative changes in the position of the US probe 12. The inertial sensor 30 can be a gyroscope, an accelerometer, or any other suitable sensor that tracks position of the US probe 12.

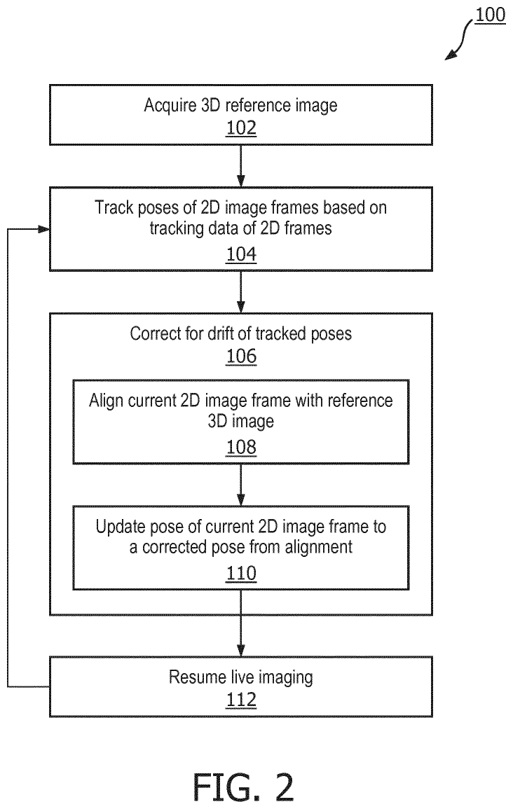

With reference to FIG. 2, an illustrative embodiment of the drift correction method 100 is diagrammatically shown as a flowchart. To start the process, the US probe 12 is positioned on or near to a portion of the patient to be scanned (e.g., the abdomen of the patient, inside a rectum in the case of transrectal prostrate imaging, or so forth). At 102, the at least one electronic processor 20 programmed to control the US scanner 14 and the US probe 12 to acquire a reference three-dimensional (3D) image. This may be done directly if the US sensor array 16 is capable of sonicating a 3D volume, or may be done in conjunction with a free-hand sweep of the US probe 12 performed by the surgeon. For example, during setup for surgical imaging, the display 24 may request that the surgeon perform a free-hand sweep, and the inertial sensor 30 detects when this occurs. The free-hand sweep may be a translation, or in some embodiments may be a rotation of the US probe 12 about the axis 18. After acquiring the reference 3D volume the US system thereafter acquires a succession of two-dimensional (2D) image frames at a rapid rate so as to provide live 2D imaging analogous to video. The live imaging thus tracks any movement or rotation (e.g. about axis 18) of the US probe 12 (or, more precisely, the sonicated plane 17 moves as the US probe 12 is manipulated). Preferably, the 2D images are displayed on the display device 24 so as to provide live US imaging of the interventional procedure. Optionally, the live 2D images are displayed with the reference 3D image to provide context of the portion of the patient being scanned.

At 104, during the live 2D imaging, the at least one electronic processor 20 is programmed to track a pose of each 2D image frame relative to the pose of the last 2D image frame in the succession of 2D image frames based on tracking data acquired by the tracking sensor 28. The pose refers to the spatial position and angle of the image frame in three-dimensional space. For example, the US probe 12 is shown in different positions in each image frame. In one embodiment, when the tracking sensor 28 comprises the US probe 12 and US scanner 14, the at least one electronic processor 20 is programmed to track the pose of each 2D image frame relative to the pose of the last 2D image frame in the succession of 2D image frames based on one or more detected changes of the 2D image frame relative to the last 2D image frame in the succession of 2D image frames. Changes “in-plane” are readily detected by spatially registering the current frame with the previous frame. Changes “out-of-plane” are more difficult to quantify in one known approach, the amount of speckle decorrelation is known to increase with movement of the current frame in the out-of-plane direction respective to the previous frame. Thus, a speckle decorrelation-versus-distance calibration curve may be used to quantify the out-of-plane distance the current 2D image frame has moved respective to the last 2D image frame. In other embodiments, when the tracking sensor 28 comprises the inertial sensor 30, the at least one electronic processor 20 is programmed to track the pose of each 2D image frame relative to the pose of the last 2D image frame in the succession of 2D image frames based at least in part on the relative changes in the position of the US probe 12 tracked by the inertial sensor. A combination of imaging-based tracking and inertial sensor-based tracking is contemplated to provide improved accuracy over either technique alone. For example, accurate imaging-based in-plane motion tracking may be combined with inertial tracking of out-of-plane motion.

The tracking performed at 104 is relative, in the sense that the pose of each 2D image frame is tracked relative to the pose of the last 2D image frame in the succession of 2D image frames. As a consequence, any errors in the pose of the current frame estimated respective to the last frame will generally accumulate over time. Such cumulative error is particularly likely to be problematic when the US probe 12 is moved over large distances or in a complex way (e.g. a combination of a translational sweep and a rotation), and when the duration of the imaging is long. This cumulative error is also referred to herein as “drift”.

Accordingly, at 106, the at least one electronic processor 20 is programmed to correct for drift of the tracked poses of the succession of 2D image frames by drift correction operations. In some examples, the drift correction operations can be triggered by manual activation of a user input (e.g., via the at least one user input device 22). In other examples, the at least one electronic processor 22 is programmed to trigger the drift correction operations by detecting rotation of the US probe 12 using the tracked poses of the succession of 2D image frames. This rotation data is measured by the tracking sensor 28. In further examples, the at least one electronic processor 22 is programmed to trigger the drift correction operations by determining when the similarity metric is outside a range of a correction threshold. The similarity metric can also be frame to frame correlation between the acquired 2D frame and the reference frame from the 3D volume, such as a correlation value of 1 indicates two identical frames and 0 indicates two uncorrelated frames. In this case, once the correlation value is lower than, for example, 0.8 between the acquired 2D frame and reference frame obtained from the 3D volume through tracking the drift correction can be triggered. Alternatively, the trend of this correlation value can be analyzed. For example, although lower values are not desirable for a steady value of 0.8 for several frame-to-frame correlations indicate some initial drift, which is not accumulating. However if the correlation value is rapidly decreasing from 0.99 to 0.8 in a few frames this indicates a more severe drift and needs to be corrected immediately therefore the drift correction can be triggered. The drift correction operations 106 can include an aligning operation 108 and an updating operation 110.

At 108, the at least one electronic processor 20 is programmed to align a current 2D image frame with the reference 3D image to optimize a similarity metric measuring similarity of the current 2D image frame to an intersected slice of the reference 3D image that was acquired at step 102. By way of non-limiting illustrative example, similarity metrics that can be used include sum of squared differences (SSD), mutual information (MI), correlation coefficient (CC), the threshold and the rate of change of the frame to frame correlation, etc. In some examples, an absolute threshold of 0.8 works for a CC. In other examples, for SSD and MI, the absolute values of these metrics depend on the image contents (and is not bound to [0 . . . 1]) and thus no pre-defined absolute threshold exists. Instead, a threshold based on the change relative to the theoretical maximum for the individual metric can be used, with the maximum being the value of the metric calculated for the last 2D image frame itself. A 20% decline (i.e. down to 0.8 times the maximum) can be used as threshold to trigger the compensation.

At 110, the at least one electronic processor 20 is programmed to update the pose of the current 2D image frame to a corrected pose determined from the alignment of the current 2D image frame with the reference 3D image. In some embodiments, the pose of the current 2D image frame tracked relative to the pose of the last 2D image frame in the succession of 2D image frames is used as an initial pose estimate for aligning the current 2D image frame with the reference 3D image. For a succession of 2D image frames, the pose of the 2D image frame following the current 2D image frame in the succession is tracked relative to the updated pose of the current 2D image frame.

At 112, the at least one electronic processor 20 is programmed to then resume with the live imaging with processing flowing back to 104 to continue tracking, but starting now from the updated pose generated at 110.

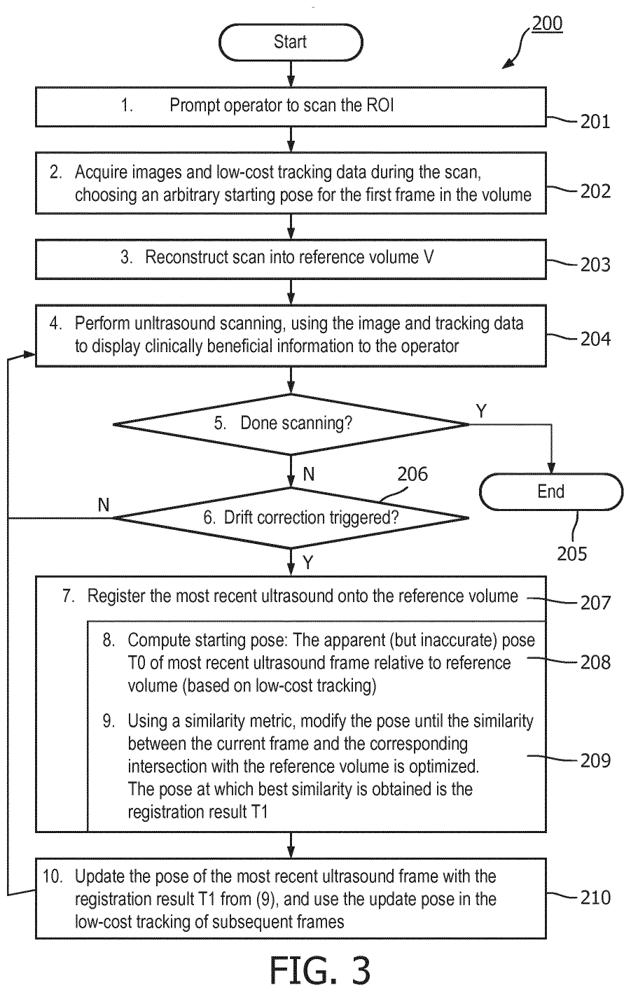

FIG. 3 is a flow chart showing a method 200 describing one illustrative implementation of the operations 102-112 in more detail. Steps 201, 202, and 203 correspond to operation 102 of FIG. 2, i.e. acquiring the reference 3D image. At 201, the US imaging device 10 is configured to prompt the operator (e.g., doctor, nurse, technician, and the like) to scan a region of interest (ROI) (e.g. the organ of the patient that is to be assessed or in which an ultrasound procedure is to be performed, such as the prostate). The prompt can be a visual prompt (e.g., displayed on the display device 24), an audible prompt (e.g., via a speaker (not shown)), or a haptic prompt (e.g., the probe 12 can vibrate).

At 202, the at least one electronic processor 20 is programmed to receive imaging data from the US probe 12 and tracking data from the inertial sensor 30. The at least one electronic processor 20 is programmed to assign a pose estimate to each image frame. For example, an arbitrary pose is assigned to the first frame, and the pose of subsequent frames is determined relative to the pose of the first frame.

At 203, the at least one electronic processor 20 is programmed to reconstruct the US images obtained during the scan based on the pose estimates. The individual US images are reconstructed into a 3D US reference volume.

At 204, the live imaging commences. The at least one electronic processor 20 is programmed to control the display device 24 to display the obtained 2D image frames and the pose estimates. The method 200 continues to either operation 205 or 206.

At 205, the method 200 is terminated. This may possibly occur without ever triggering a drift correction if the imaging session is short and/or the US probe 12 is not moved during the session. However, for longer sessions and/or sessions with substantial movement of the US probe 12, one or more drift corrections may be performed over the course of the live imaging of the interventional procedure.

To this end, at 206, a drift correction is triggered. The drift correction can be triggered in various ways, such as by: (1) a regular time interval (e.g., every ten seconds); (2) a user input received via the user input device 22 (e.g., a mouse click, a keyboard button press, and the like); (3) automatically when tracking accuracy is likely low (e.g. a similarity between current frame and corresponding slice of the reference volume is low); or (4) the at least one electronic processor 20 is programmed to detect significant rotations of the US probe 12 from the tracking data obtained from the tracing sensor 28 that are known to reduce the accuracy of the low-cost tracking (e.g.: rotations around the probe axis with angles near 90 degrees, such as required to switch from axial views to sagittal views in prostate scanning) A given implementation may employ any one, any two, or more, or all of these drift correction triggering mechanisms.

At 207, the at least one electronic processor 20 is programmed to perform drift correction operations 208 and 209. The drift correction can include a re-registration of the current US frame to the 3D reference volume. At 208 the at least one electronic processor 20 is programmed to compute a starting pose Tₒ of the current frame in the 3D reference volume. The starting pose is computed from data received from the tracking sensor 28. At 209, the at least one electronic processor 20 is programmed to modify or adjust the pose of the current frame relative to the reference volume until the similarity between the current frame and the corresponding section in the reference volume is optimized to generate an optimized pose T₁. Similarity metrics that can be used include sum of squared differences (“SSD”), mutual information (“MI”), correlation coefficient (“CC”) etc. In some examples, a predefined number of images (e.g., thirty) can be used for registration instead of just the most recent frame, in order to enhance the robustness of the 3D reference volume.

At 210, the at least one electronic processor 20 is programmed to use the optimized pose T₁ as the new or updated pose for the current frame. The optimized pose T₁ is used as the updated pose estimate for next iteration of operation 204 of the live imaging.

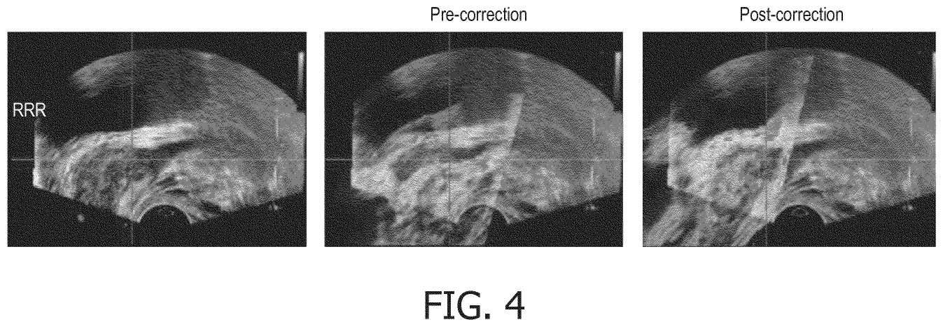

FIG. 4 shows an example of the drift correction based on optimizing the similarity between the current frame and the reference volume. The current frame only is shown on the left, the current frame fused with the reference volume (color) based on the inaccurate pose T₀ is shown in the center, and the fused images based on the optimized pose T₁ is shown on the right.

The disclosure has been described with reference to the preferred embodiments. Modifications and alterations may occur to others upon reading and understanding the preceding detailed description. It is intended that the disclosure be construed as including all such modifications and alterations insofar as they come within the scope of the appended claims or the equivalents thereof.

1. An ultrasound (US) device, comprising:
a US scanner and a US probe operatively connected to the US scanner, the US scanner and US probe configured to acquire a succession of two-dimensional image frames of a portion of a patient;
a tracking sensor; and
at least one electronic processor programmed to:
acquire a reference three-dimensional image using the US scanner and US probe;
track the pose of each 2D image frame relative to the pose of the last 2D image frame in the succession of 2D image frames based on tracking data acquired by the tracking sensor; and
correct for drift of the tracked poses of the succession of 2D image frames by drift correction operations including:
aligning a current 2D image frame with the reference 3D image to optimize a similarity metric measuring similarity of the current 2D image frame to an intersected slice of the reference 3D image; and
updating the pose of the current 2D image frame to a corrected pose determined from the alignment of the current 2D image frame with the reference 3D image.

2. The device of claim 1, wherein the pose of the 2D image frame following the current 2D image frame in the succession is tracked relative to the updated pose of the current 2D image frame.

3. The device of claim 1, wherein the drift correction is triggered by manual activation of a user input.

4. The device of claim 1, wherein the at least one electronic processor is further programmed to:
trigger the drift correction operations by detecting rotation of the US probe using the tracked poses of the succession of 2D image frames.

5. The device of claim 1, wherein the at least one electronic processor is further programmed to:
trigger the drift correction operations by determining when the similarity metric is outside a range of a correction threshold.

6. The device of claim 1, wherein the tracking sensor comprises the US scanner and US probe; and
the at least one electronic processor is programmed to track the pose of each 2D image frame relative to the pose of the last 2D image frame in the succession of 2D image frames based on one or more detected changes of the 2D image frame relative to the last 2D image frame in the succession of 2D image frames.

7. The device of claim 1, wherein the tracking sensor comprises an inertial sensor tracking relative changes in the position of US probe; and
the at least one electronic processor is programmed to track the pose of each 2D image frame relative to the pose of the last 2D image frame in the succession of 2D image frames based at least in part on the relative changes in the position of the US probe tracked by the inertial sensor.

8. The device of claim 7, wherein the inertial sensor comprises at least one of a gyroscope or an accelerometer.

9. The device of claim 1, wherein the pose of the current 2D image frame tracked relative to the pose of the last 2D image frame in the succession of 2D image frames is used as an initial pose estimate for aligning the current 2D image frame with the reference 3D image.

10. The system of claim 1, wherein the at least one electronic processor is further programmed to:
control a display device to display the drift correction operations.

11. A non-transitory computer readable medium storing instructions executable by at least one electronic processor to perform a drift correction method, the method comprising:
acquire two-dimensional images using a US scanner and a US probe;
acquire a reference three-dimensional image using the US scanner and the US probe;
track a pose of each 2D image frame relative to the pose of the last 2D image frame in the succession of 2D image frames based on tracking data acquired by a tracking sensor; and
correct for drift of the tracked poses of the succession of 2D image frames by drift correction operations including:
aligning a current 2D image frame with the reference 3D image to optimize a similarity metric measuring similarity of the current 2D image frame to an intersected slice of the reference 3D image; and
updating the pose of the current 2D image frame to a corrected pose determined from the alignment of the current 2D image frame with the reference 3D image.

12. The non-transitory computer readable medium of claim 11, wherein the pose of the 2D image frame following the current 2D image frame in the succession is tracked relative to the updated pose of the current 2D image frame.

13. The non-transitory computer readable medium of claim 11, wherein the method further includes:
triggering the drift correction operations by determining when the similarity metric is outside a range of a correction threshold.

14. The non-transitory computer readable medium of claim 11, wherein the tracking sensor comprises an inertial sensor tracking relative changes in the position of US probe, and the method further includes:
tracking the pose of each 2D image frame relative to the pose of the last 2D image frame in the succession of 2D image frames based at least in part on the relative changes in the position of the US probe tracked by the inertial sensor.

15. (canceled)

16. (canceled)

17. (canceled)

18. (canceled)

19. (canceled)

20. (canceled)
